(19) United States
(12) Patent Application Publication
(10) Pub. No.: US 2008/0081368 A1
(43) Pub. Date: Apr. 3, 2008

(54) Method and apparatus that increases efficiency and reproducibility in immunohistochemistry and immunocytochemistry

(76) Inventors: Anne Marie Bailey, Columbia, SC (US); Lorne John Hofseth, Columbia, SC (US)

Correspondence Address: Anne Marie Bailey ; Lorne John Hofseth, 163 Williamstown Way, Columbia, SC, 29212, US

(21) Appl. No.: 11/906,215

(22) Filed: Oct. 1, 2007

Related U.S. Application Data
(60) Provisional application No. 60/849,117, filed on Oct. 3, 2006.

(51) Int. Cl. C12N 5/06; C12M 1/02

(52) U.S. Cl. 435/325; 435/307.1

(57) ABSTRACT

A method combined with its necessary container package that facilitates unique processing of microscope slides in a reagent solution is disclosed. This invention has not been described elsewhere. The container package is a partitioned box with each compartment fitting one standard microscope slide. The slides are immersed in a staining solution and the container package is covered with a lid. The container package and slides/staining solution are placed on a standard laboratory apparatus and rocked or agitated for an arbitrary incubation time. The method and its necessary container package provides a means for more reproducibility and sensitivity of results than the prior art that is critical for accurate scientific findings.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The staining and examining of biologic specimens occurs daily in great numbers in hospital and scientific laboratories, medical and veterinary clinics. Current techniques rely on a sedentary approach whereupon a plurality of staining fluid is placed over a biological specimen and incubated for a said period of time while remaining sedentary. This technique can lead to drying of tissues if the staining fluid does not cover all the specimens, and pooling of the stain resulting in uneven staining. To stain a biologic specimen fixed on a glass slide, it is necessary to bring a staining liquid or reagent into contact with the specimen on the slide. For the present purposes it will be understood that the term staining includes application to the specimen of any of various liquids or reagents during a staining process, even if a particular liquid does not itself produce the actual staining. Two techniques used for staining specimens mounted or attached to glass microscope slides are immunohistochemistry and immunocytochemistry.

Immunohistochemistry requires a sequence of steps as follows: 1) removing of embedding or stabilizing material from mounted tissue on microscope slides; 2) rehydration of mounted tissue; 3) washing of mounted tissue; 4) unmasking of cellular epitopes; 5) blocking of tissue samples; 6) incubation of mounted tissue with desired antibody; 7) secondary washing of mounted tissue; 8) incubation of tissue with secondary antibody and 9) addition of detection compound. This is a generalization of a technique that is used to analyze proteins in tissue samples, as there are varied ways of preserving tissue, for example, by freezing, or submerging in paraffin wax.

As mentioned, current techniques rely on a sedentary approach whereupon a plurality of staining fluid is placed over a biological specimen and incubated for a said period of time while remaining sedentary. This technique can cause drying of tissues if the staining fluid does not cover all the specimens, and pooling of the stain resulting in uneven staining.

Developing methods and/or apparatus' that overcome the issues of antibody pooling and tissue drying would be welcomed by the scientific community. Current inventions address common issues, such as the labor intensive nature of staining biological specimens, by patenting automated staining techniques (U.S. Pat. No. 7,025,937; U.S. Pat. No. 6,436,348; U.S. Pat. No. 6,017,495). However, such inventions (e.g. U.S. Pat. No. 7,025,937) do not overcome issues such as tissue drying, antibody pooling, and inconsistent staining because of the sedentary techniques used during incubation. Furthermore, the large volumes of liquid required for other automated techniques (e.g. U.S. Pat. No. 6,436,348; U.S. Pat. No. 6,017,495) make it financially infeasible to use for immunohistochemical and/or immunocytochemical staining, where expensive antibodies are used. Although other inventions use low volumes, they do not overcome some problems present with systems requiring capillary action of fluid between two slides, especially when viscous reagents must be used. Additionally, there is still risk for tissue drying, antibody pooling, and inconsistent staining associated with the sedentary nature of incubation (see U.S. Pat. No. 4,635,791; U.S. Pat. No. 5,002,736; U.S. Pat. No. 4,777,020; U.S. Pat. No. 4,985,206; and U.S. Pat. No. 5,192,503). Chu (U.S. Pat. No. 5,958,341) describes an apparatus requiring low volumes of fluid, and placing the tissue down toward a well containing the stain. Although this method addresses issues such as tissue drying, and antibody pooling, it also relies on capillary action to stain tissues, which may be associated with uneven staining. Furthermore, the sedentary nature of the incubation suggests the antibody dilution would remain similar to the standard dropping method and associated sedentary incubation. Finally, pulling the tissue up from the stain/liquid following incubation increases the risk of suctioning off part or all of the tissue, therefore compromising the integrity of the tissue.

Immunocytochemistry is a technique used to analyze characteristics of whole cells on a slide. Live cells are seeded onto glass microscope slides, and are stained with various liquid reagents, commonly containing a labeling dye. The most frequent drawback of this technique is that the labeling reagent is pooled onto the slide, and can easily run off the slide, or stain unevenly. This method also requires transporting slides, typically from an incubator or benchtop, to a working microscope, in order to analyze cellular staining visually, which dramatically increases the risk of reagent runoff.

We propose a unique submersion rocking method combined with a multiple slide holder apparatus to overcome these drawbacks in both imunnohistochemistry and immunocytochemistry. Such a method and its necessary apparatus has not been described elsewhere. In addition to greater efficiency and enhanced reproducibility, the combination of sample submersion and rocking/agitation allows for more diluted stain, and allows for the re-use of stain without loss of sensitivity.

This current invention utilizes a method in which biological specimens are immersed in the staining solution, and rocked on standard laboratory equipment (for example, a laboratory rocker, or shaker). For clarification purposes, the term “rocked” includes any action of agitation of liquid which comes in contact with a microscope slide. This allows for the stain to be continually mixed, and the tissue will not dry out. Therefore, the combination of said apparatus and said method, which has never been described before, provides a means for more reproducible and accurate scientific findings. The dynamic nature of our invention ensures even staining throughout the tissue. This process of rocking the apparatus/slide/biological specimen/stain also demonstrates that one can use much more diluted stain for staining the biological specimen. Scharf (U.S. Pat. No. 3,132,744) describes a disposable package for receiving two slides to be stained. The package is filled with a staining liquid prior to use, and the slides are immersed in the pool of liquid in order to stain the specimens. Although the process of immersion described in this patent is also described here-in, Scharf does not describe the method of placing the apparatus on a laboratory rocker/shaker to enhance staining consistency. Several other disadvantages of the Scharf invention include that only 2 slides can be stained per apparatus, that a relatively large quantity of expensive staining liquid is used for the immersion and staining of the two slides, and that the centrally extending ribs which separate the two slides can disturb a specimen affixed to the face of a slide.

One of the more recent and widely used technologies used by people trained in the art involves a complex system (U.S. Pat. No. 5,958,760). This system allows liquids to fill a gap between the coverplate and slide. The liquid stays in the gap until the next reagent is dispensed into the well. Its pressure then causes the previous reagent to be displaced by gravity flow. This process claims to protect specimens from drying during long incubations. Although consistency in staining quality is claimed, our said container package with said method of rocking/shaking ensures not only consistency of staining quality, but increases sensitivity, as well as ensuring the specimens do not dry out. Said container package with said method has not been described elsewhere.

### BRIEF SUMMARY OF THE INVENTION

The present invention relates to a container package partitioned to hold a plurality of individual standard laboratory slides and be secured with a removable lid. This design allows said slides to be rocked on laboratory equipment, and transported with ease. Said apparatus and technique results in no antibody pooling, nor tissue drying commonly encountered in standard sedentary immunohistochemical/immunocytochemical techniques. There is no risk for runoff with this combination of apparatus and method. This combination of apparatus and method provides stable transport for liquid immersed slides during techniques used for immunohistochemistry/immunocytochemistry. This results in better consistency, and increases the sensitivity of the assays. The base of the container package comprises partitions that divide said container package into individual compartments. Each individual compartment holds one standard laboratory slide, with a biological specimen affixed to the slide. The container package comprises a lid of the same or similar material as the base apparatus, so as to reduce evaporation, encourage stacking of the apparatus, and finally, give ease of storage and transport.

When one or a plurality of standard laboratory slides are placed in said container package, solution is placed into said compartment; enough to cover said slide. Said base of the container package is covered by said lid of the container package, and said apparatus can be rocked/shaken on standard laboratory equipment.

### BRIEF DESCRIPTION OF THE DRAWINGS

FIG. 1 is a top view of the base container package, lid off

FIG. 2 is a side sectional view of FIG. 1, lid off

FIG. 3 is an end sectional view of FIG. 1, lid off

FIG. 4B is a cross sectional view of FIGS. 2 and 4A, lid off

FIG. 5 is a top view of the top side of the container package lid

FIG. 6 is a top view of the bottom side of the container package lid

FIG. 7 is a side sectional view of the container package lid

FIG. 8 is an end sectional view of the container package lid

FIG. 9 is a side sectional view of the container package base with container package lid

FIG. 10 is an end sectional view of the container package base with container package lid

FIG. 11 is an immunohistochemical stain showing the method of rocking the container package greatly increases the sensitivity of the assay, which saves money.

FIG. 12 is an immunohistochemical stain showing the method of rocking the container package promotes even staining.

FIG. 13 is an immunohistochemical stain showing the method of rocking the container package greatly increases the reproducibility of the assay.

FIG. 14 is an immunohistochemical stain showing the method of rocking the container package allows the stain to be re-used several times, which saves money.

FIG. 15 are examples of typical immunohistochemical stains showing the method of rocking the container package can be used with different antibodies, not limited to those shown here.

### DETAILED DESCRIPTION OF THE INVENTION

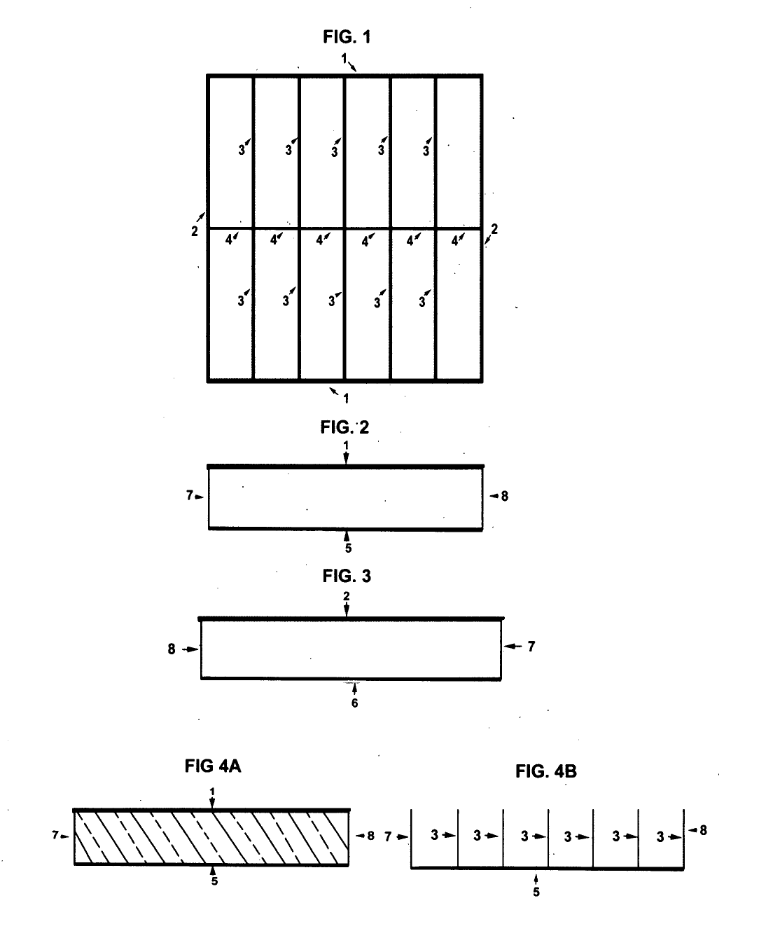

FIGS. 1, 2, 3 illustrates the top, side and end views of the container package base, without the container package lid. FIG. 4B is a cross sectional view of FIGS. 4A and 2. Said container package base has a bottom 5 and 7, that has walls outlined by 1, 5, 7, and 8 (FIG. 2), and 2, 6, 7, and 8 (FIG. 3). The container package base is partitioned into compartments by 3 and 4 (FIG. 1 and FIG. 4B). The compartments are preferably large enough to fit one standard laboratory microscope slide. FIG. 2 illustrates the side view of the container package base, with the wall outlined by 1, 5, 7 and 8. FIG. 4B illustrates a cross sectional view of FIG. 4B of the container package base, such that the compartments are visible.

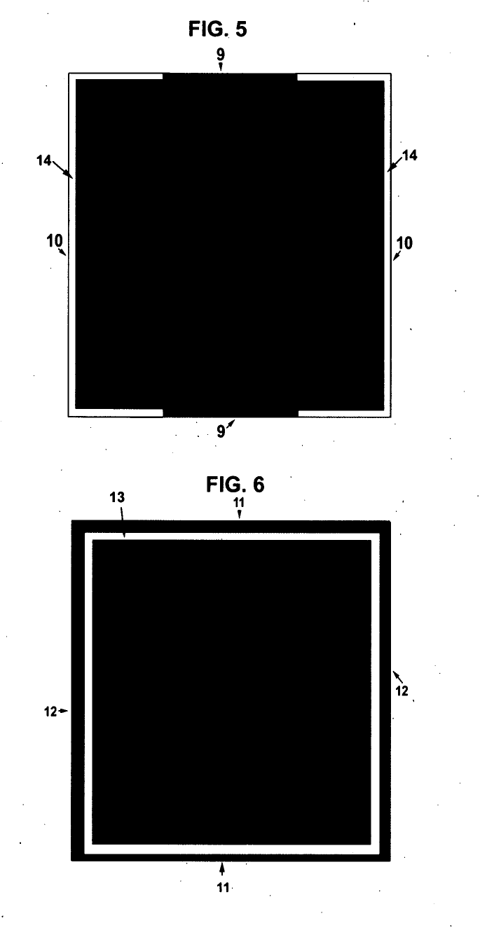

FIGS. 5 and 6 illustrate a top view of the top side, and the bottom side of the container package lid. The top side (FIG. 5) has an elevated lip 14 within which the bottom 5 and 7 fit into to allow for stacking of said container packages. The bottom side (FIG. 6) has an elevated lip 13 that allows the base of the container package (FIG. 1) to fit into, giving a tight seal between the lid and the base of the container package. This tight seal reduces risk of evaporation of said liquid.

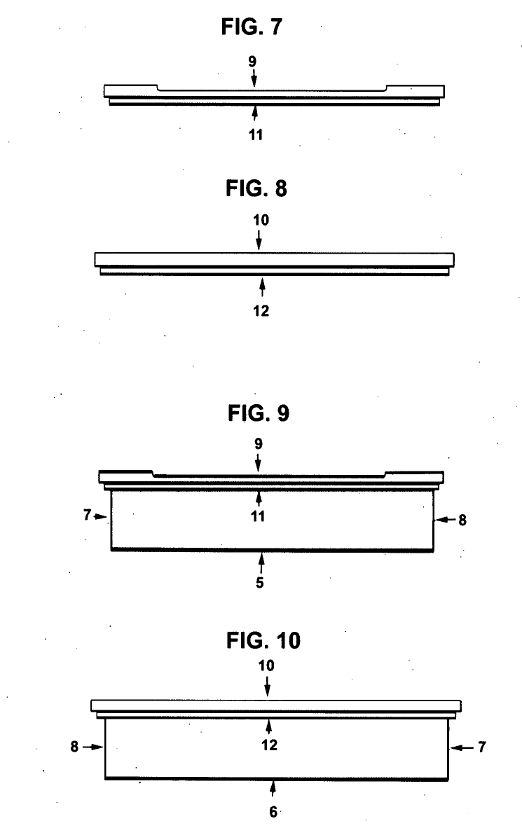

FIGS. 7 and 8 illustrate the side and end sectional views of the container package lid. Said container package lid has a bottom 11 and 12 and top 9 and 10. FIGS. 9 and 10 illustrate said container package base with container package lid.

In the preferred embodiment, the material of said container package base and container package lid is of a polypropylene nature that has a heat deflection temperature of 140° C. or above. This is necessary to withstand autoclaving and/or microwaving processing of tissues that is associated with processing tissues in some procedures that said container package can be used in. Said container package (base and lid) are easy to manufacture using plastic injection molding techniques at a reasonable price. This ensures wide-spread availability to the average scientist who is seeking a way to increase their confidence in immunohistochemical/immunocytochemical staining.

The invention here-in involves the practice of rocking/shaking biological specimens affixed to standard laboratory microscope slides in a staining solution that will often contain antibodies or other biological probes to stain said tissues. This practice of rocking/shaking requires said container package base and lid outlined in FIGS. 1-10 above. This practice of rocking biological specimens in said container package increases the sensitivity of the assay. Furthermore, this practice circumvents common problems associated with immunohistochemical staining that includes uneven staining and non-reproducible results.

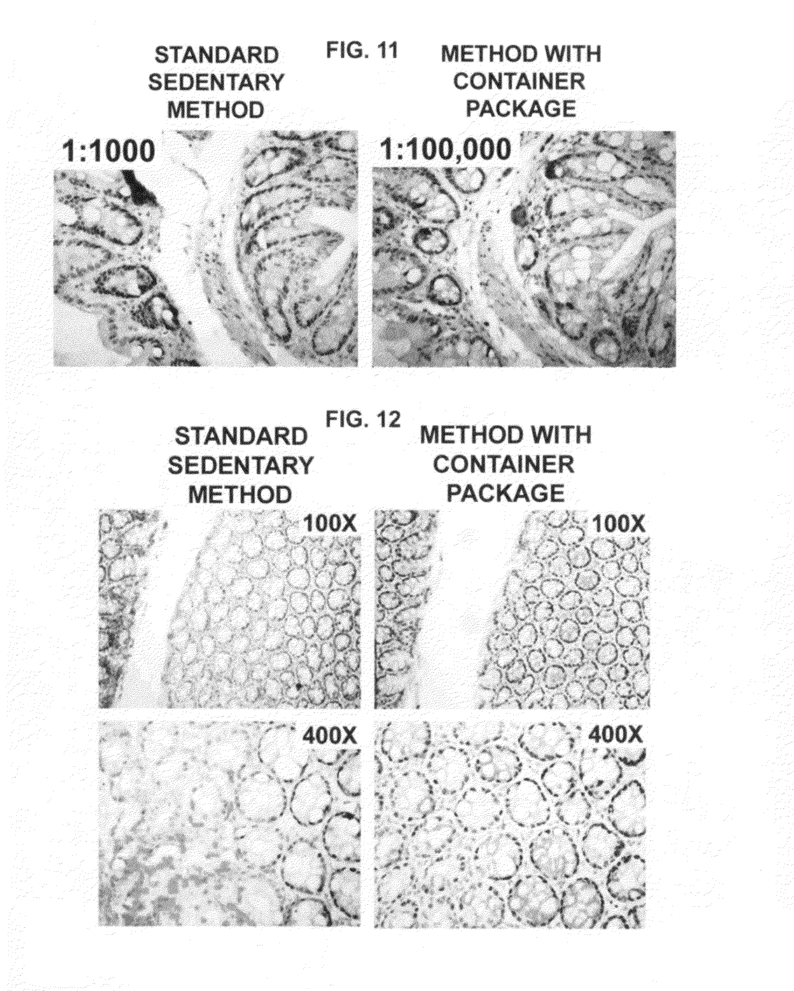

FIG. 11 illustrates an immunohistochemical stain showing the method of rocking/shaking the container package greatly increases the sensitivity of the assay, which saves money for individuals trained in the art. The antibody used here detects phosphorylated retinoblastoma protein, and the tissue used was mouse colon. The colon tissues were from serial sections. The antibody concentration used for the standard sedentary method was 1:1000. The antibody concentration used for the method of rocking said slide/tissue/antibody solution/container package was 1:100,000.

FIG. 12 illustrates an immunohistochemical stain showing the method of rocking the container package promotes even staining. The antibody used here detects phosphorylated retinoblastoma protein, and the tissue used was mouse colon. The colon tissues were from serial sections. Photomicrographs were taken at 100× and 400× magnification as indicated under a light microscope. The antibody concentration used for the standard sedentary method was 1:1000. The antibody concentration used for the method of rocking said slide/tissue/antibody solution/container package was 1:100,000.

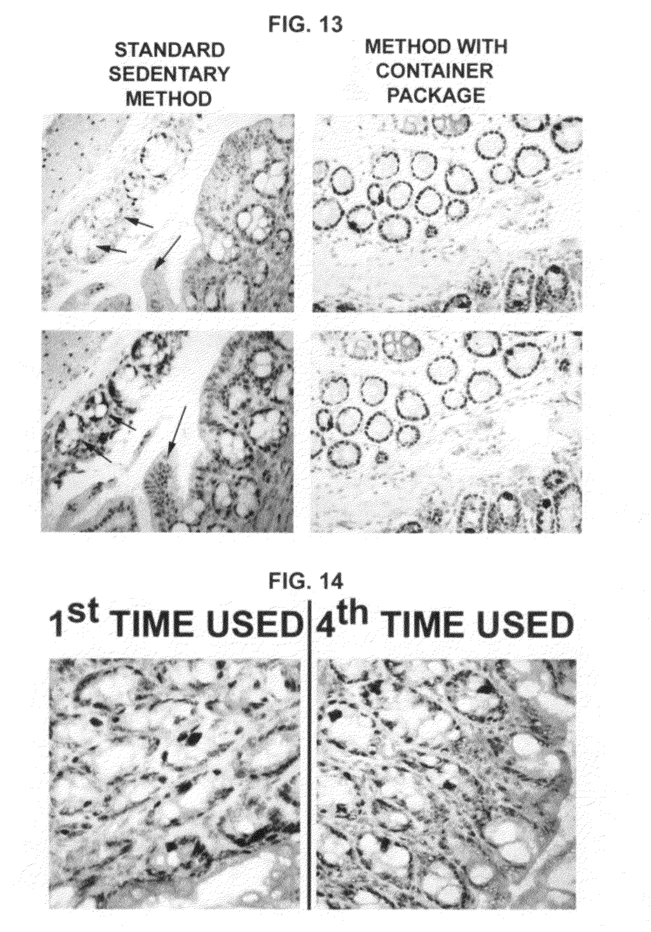

FIG. 13 illustrates an immunohistochemical stain showing the method of rocking the container package greatly increases the reproducibility of the assay. The antibody used here detects phosphorylated retinoblastoma protein, and the tissue used was mouse colon. For the standard sedentary method serial sections were stained on separate days using the same methods. For the method of rocking said slide/tissue/antibody solution/container package, serial sections were also stained on separate days using the same methods. Arrows indicate where staining is weak on the first section, but darker on the second serial section, even though the identical techniques were used to stain said tissues. This does not occur when using said method of rocking said slide/tissue/antibody solution/container package. The antibody concentration used for the standard sedentary method was 1:1000. The antibody concentration used for the method of rocking said slide/tissue/antibody solution/container package was 1:100,000.

FIG. 14 illustrates an immunohistochemical stain showing the method of rocking the container package allows the stain to be re-used several times, which saves money. Although this practice is sometimes used, those trained in the art generally believe there is reduced sensitivity of the stain each time it is used and frozen during storage. FIG. 14 illustrates the stain is equally sensitive even after using and freezing said stain up to four times. The antibody concentration used was 1:100,000.

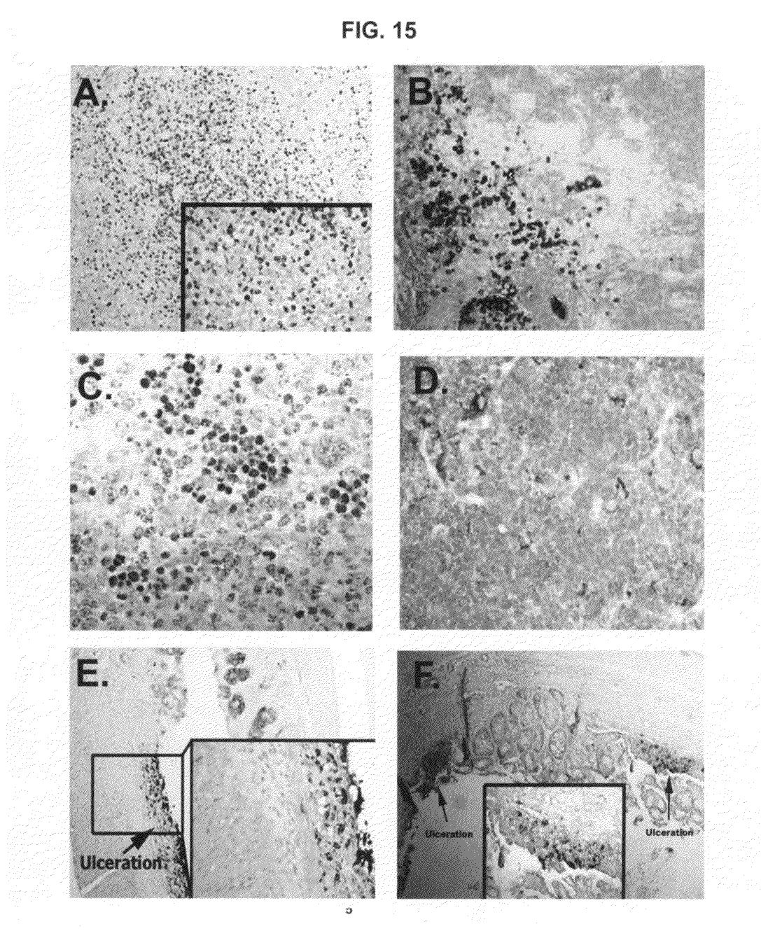

FIG. 15 illustrates examples of typical immunohistochemical stains showing the method of rocking/shaking the container package can be used with different antibodies, not limited to those shown here. FIG. 15A shows staining of human tonsil, using anti-MIB-1 antibody, which detects a proliferation marker called Ki67. The antibody was used at 13 fold lower dilution than that recommended by the company. FIG. 15B shows staining of mouse thymus, using anti-p21 antibody, which detects a molecule involved in cell cycle. The antibody was used at 80 fold lower dilution than that recommended by the company. FIG. 15C shows staining of mouse thymus, using anti-FAS-L antibody, which detects cell death. The antibody was used at 2.5 ug/ml, and the company does not recommend use in immunohistochemistry. The finding that the antibody can be used with said technique of rocking said slide/tissue/stain/container package indicates a superior method for immunohistochemistry procedures. FIG. 15D shows staining of mouse spleen, using anti-TRAIL antibody, which detects a molecule involved in cell death. The antibody was used at 200 fold lower dilution than that recommended by the company. FIG. 15E shows staining of mouse colon, using anti-COX-2 antibody, which detects cyclooxygenase-2, a molecule involved in inflammation. FIG. 15E shows darker staining in areas of colon ulceration, where inflammation is active. FIG. 15F shows staining of mouse colon, using anti-NOS-2 antibody, which detects nitric oxide synthase-2, a molecule involved in inflammation. FIG. 15F shows darker staining in areas of colon ulceration, where inflammation is active.

An example of the immunohistochemical method used with the container package is as follows:

1.) The container package contains a pleurality of wells, and therefore holds a pleurality of microscope slides. Each well contains one standard microscope slide with a biological specimen fixed to each slide.

2.) Deparaffinize slides (optional to use container package)

3.) Wash (optional to use container package).

4.) Antigen retrieval (optional to use container package)

5.) Incubation of slides with primary antibody using the container package. Although 3 milliliters is required to submerge each slide, dilutions of primary antibody of up to 200 times more than the standard dilution have been used. Agitate or rock the container on standard laboratory equipment for desired time.

6.) Wash (optional to use the container package).

7.) Incubation of slides with secondary antibody. Although 3 milliliters is required to submerge the slides, dilutions of secondary antibody up to 100 times more than the standard dilution have been used.

8.) Wash

9.) Substrate addition

10.) Counterstain.

What we claim is:

1. A combination of method and required container package for unique processing of biological samples that may typically involve a) the submerging of organ tissues or cells mounted on a microscope slide in an organic solvent b) rehydrating said tissue or cells in organic reagent or reagents c) performing detergent washes on said fixed tissue or cells at various determined times d) exposing said tissue or cells to liquid blocking agents e) incubation requiring rocking or agitating said tissue or cells in an apparatus designed for holding a plurality of microscope slides submerged in a minimal volume of primary labeling reagent or liquid f) storing said antibody post incubation for future use g) exposing tissue or cells to secondary labeling reagent h) staining and counterstaining said tissue or cells with inorganic compound or compounds.

2. That, in a method and apparatus for processing biological samples, biological samples on a microscope slide or slides are submerged under a primary labeling reagent or reagents in the heat-resistant apparatus described above, and rocked or agitated for a period of time;

3. A method and apparatus for processing samples in immunohistochemistry and immunocytochemistry requiring the incubation and/or transportation of microscope slides in a watertight, autoclavable container capable of immersing a microscope slide in a minimal amount of solution;

4. The device according to claim three wherein the apparatus is covered with a lid that provides either single or multiple openings, that also is comprised of the same or similar material as the apparatus so as to reduce evaporation, encourage stacking of the apparatus, and finally, give ease of storage;

5. The device according to claim three wherein the apparatus can hold a plurality of microscope slides;

6. The device according to claim three wherein the apparatus is made of a material capable of withstanding the autoclaving process;

7. A method of incubation using the apparatus in claim three where the labeling reagent or reagents can be reused;

8. An apparatus in claim three that has separate watertight compartments so to allow the use of separate labeling reagents.
